(19) United States
(12) Patent Application Publication
(10) Pub. No.: US 2022/0062418 A1
(43) Pub. Date: Mar. 3, 2022

(54) TUMOR TARGETING VITAMIN B12 DERIVATIVES FOR X-RAY ACTIVATED CHEMOTHERAPY

(71) Applicant: THE TRUSTEES OF DARTMOUTH COLLEGE, Hanover, NH (US)

(72) Inventors: Jennifer SHELL, South Royalton, VT (US); Brian POGUE, Hanover, NH (US)

(21) Appl. No.: 17/298,944

(22) PCT Filed: Nov. 27, 2019
(86) PCT No.: PCT/US2019/063790

§ 371 Date: Jun. 1, 2021

Related U.S. Application Data
(60) Provisional application No. 62/773,097, filed on Nov. 29, 2018.

(51) Int. Cl. A61K 41/00; A61K 47/55; A61K 49/00; A61K 31/704; A61K 31/337; A61K 31/519; A61K 31/517; A61K 31/196; A61K 31/506; A61K 31/165; A61K 31/5377; A61K 31/4745; A61K 38/17

(52) U.S. Cl. CPC A61K 41/0042; A61K 47/551; A61K 49/0052; A61K 31/704; A61K 31/337; A61K 31/519; A61K 38/1709; A61K 31/196; A61K 31/506; A61K 31/165; A61K 31/5377; A61K 31/4745; A61K 31/517

(57) ABSTRACT

A therapeutic agent has an antineoplastic drug bonded with an X-ray-cleavable bond to cobalt of cobalamin. In embodiments, the drug is doxorubicin, paclitaxel, methotrexate, erlotinib, chlorambucil, dasatinib, SN38, colchicine, or gefitinib; and in embodiments a Cy5 fluorophore bonded to ribose of the cobalamin. The agent is formed by reducing hydroxocobalamin with zinc, reacting with 3-bromopropylamine to form aminopropyl cobalamin; and linking the drug to the aminopropyl cobalamin by conjugation through a hydroxyl group by carbamate formation with 1, 1′-Carbonyl-di-(1,2,4-triazole). An optional Cy5 handle is added by coupling a 5′ hydroxyl group of a ribose first with ethylene diamine and then with N-hydroxysuccinimide of Cy5. The agent treats cancer by administration in a dose expected to induce apoptosis in cells of the cancer when the light-cleavable bond is cleaved, the cancer absorbs the agent; and the cancer is exposed to X-ray or visible light to cleave the X-ray-light-cleavable bond.

### CLAIM TO PRIORITY

The present application claims priority to U.S. Provisional Patent Application No. 62/773,097, filed Nov. 29, 2018. The entire contents of the aforementioned application are incorporated herein by reference.

### BACKGROUND

Chemotherapy is essential in treating most cancers; however, it has many side effects from hair loss and nausea to cardiomyopathy due to off-target interactions. Targeted drug delivery may reduce these side effects to off-target normal tissues, while still being effective at delivering maximal dosages to the cancer.

Light-activated delivery can target tumors by providing a spatiotemporally controlled release of an antineoplastic drug in an area of interest, such as in and around a tumor. Most light-activated drugs that have been developed are limited in that they require light wavelengths, such as shortwave ultraviolet light, that don't effectively penetrate tissue. We describe a method of chemotherapy utilizing the vitamin B₁₂ platform to deliver an X-ray-activatable drug into cells. X-ray activated phototherapy may provide a way to deliver chemotherapy synergistically with radiotherapy. X-rays provide deeper tissue penetration than the light sources utilized in traditional photodynamic therapy (PDT). X-ray activated photodynamic therapy has been performed where photosensitizers are attached to nanoscintillators. The nanoscintillators exhibit X-ray induced luminescence and in turn activate the photosensitizers in areas exposed to radiation. This in turn can provide a synergistic release of singlet oxygen in conjunction with radiation therapy, if there is efficient light transfer between the nanoscintillator and photosensitizer. However, many variables affect luminous transfer from these nanoscintillators, including the biocompatible coatings, defects in the particle, and interactions with biomolecules. Current technologies for X-ray photodynamic therapy (PDT) requiring indirect light transfer from X-ray luminescent nanoscintillators suffer from uneven X-ray excitation and therefore inconsistent drug release.

Five-year survival of pancreatic ductal adenocarcinoma (PDAC) is approximately 3%, and therefore is among the most dismal prognoses in all of oncology. Radical resection with or without adjuvant or neoadjuvant treatment is the only means of improving long-term survival, but is an option in about 25% of patients. Even when surgery is combined with chemotherapy, the disease recurs in about 80% of those patients, who die within one year of recurrence. For locally advanced and unresectable disease, a focus of patient management is preserving acceptable quality of life for as long as possible through palliative and salvage therapy. Gemcitabine is a widely used drug for PDAC; however poor uptake, among other things, limits it efficacy. PDAC remains difficult to treat due to the unusual microenvironment of these tumors, which exhibit a lack of vascularization, high stromal density, and high total tissue pressure. Drugs like gemcitabine, a cytidine analog that blocks DNA synthesis, and other cocktails used to treat PDAC suffer from significant toxicity and severe side effects due to their lack of selectivity for tumor versus normal tissue.

### SUMMARY

A therapeutic agent has an antineoplastic drug bonded with a X-ray-cleavable bond to cobalt of cobalamin. In embodiments, the drug is doxorubicin, paclitaxel, methotrexate, erlotinib, chlorambucil, dasatinib, SN38, colchicine, or gefitinib; and in embodiments a Sulfo-Cy5 fluorophore bonded to ribose of the cobalamin. The agent is formed by reducing hydroxocobalamin with zinc, reacting with 3-bromopropylamine to form aminopropyl cobalamin; and linking the drug to the aminopropyl cobalamin by conjugation through a hydroxyl group by carbamate formation with 1,1′-Carbonyl-di-(1,2,4-triazole). An optional sulfo-Cy5 handle is added by coupling a 5′ hydroxyl group of a ribose first with ethylene diamine and then with N-hydroxysuccinimide of sulfo-Cy5. The agent treats cancer by administration in a dose expected to induce apoptosis in cells of the cancer when the X-ray-cleavable bond is cleaved after the cancer absorbs the agent and the cancer is exposed to radiation

### BRIEF DESCRIPTION OF THE FIGURES

FIG. 1 illustrates structure of a cobalamin conjugated with a chemotherapeutic agent and a fluorophore in an embodiment.

FIG. 2A illustrates relative concentrations of fluorescently-labeled cobalamin conjugates (left of each bargraph pair) and unconjugated fluorophore in tissues of mice.

FIG. 2B illustrates relative concentration of fluorescently-labeled cobalamin conjugates in normal muscle (lower line) and tumor tissues (upper line) of mice.

FIG. 3A illustrates release, and activation, of drug under light exposure.

FIG. 3B illustrates composition of a cobalamin-drug conjugate.

FIG. 3C illustrates 400-580 nm. photon-triggered decomposition of the cobalamin-drug conjugate of FIG. 3B.

FIG. 4 illustrates an increase in fluorescence as drug and/or fluorophore is released under light exposure, as tested with a Bdy-Cbl conjugate measured with an excitation wavelength of 640 nm and emission wavelength of 680 nm.

FIG. 5 illustrates an increase in fluorescence as drug and/or fluorophore is released under X-ray exposure, as tested with a Bdy-Cbl conjugate.

FIG. 6 illustrates a cobalamin-doxorubicin conjugate that has been synthesized, shown to be selectively absorbed by tumor cells overexpressing cobalamin receptors, and to release the drug doxorubicin upon light exposure.

FIG. 7 illustrates lethality of light or X-ray-activated Cbl-Erlotinib conjugates in mouse pancreatic tumor cells.

FIG. 8 illustrates a synthesis of Cbl-SN38 and Cbl-Erl.

FIG. 9 illustrates structures of paclitaxel (Tax) and methotrexate (Mth) with points of bonding to the cobalt of cobalamin indicated with wavy lines.

FIG. 10 is a flowchart illustrating a method of treatment of a cancer.

### DETAILED DESCRIPTION OF THE EMBODIMENTS

We advance a combination targeted approach to therapy which injects chemotherapeutic agents into the tissue at the point treatment is needed, using as a conduit a cobalamin scaffold that is subject to the body's natural uptake mechanism for vitamin B12.

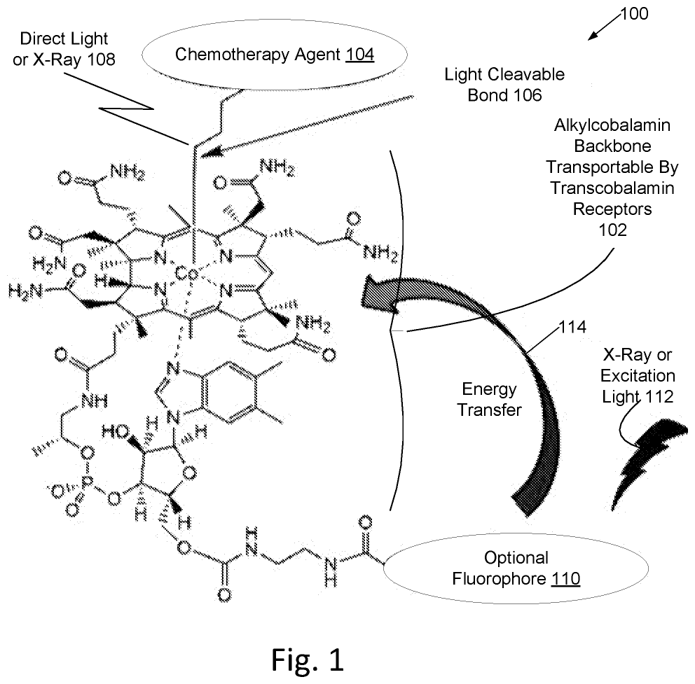

Our cobalamin-based scaffold 102 (FIG. 1) is linked to a chemotherapeutic agent 104 by a light-cleavable bond 106 to the cobalt of the cobalamin. The vitamin B12 platform allows for selective uptake of cobalamin-drug conjugate into tumors, since a variety of cancers overexpress transcobalamin receptors. These overexpressed transcobalamin receptors (TBclR) provide a way to ferry the cobalamin-drug conjugates 100 into cancer cells disguised as vitamin B12, followed by release of drug cargo upon X-ray irradiation at the tumor site.

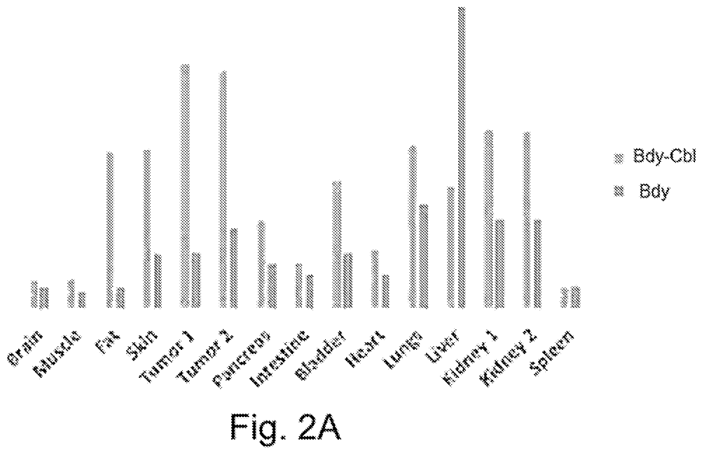

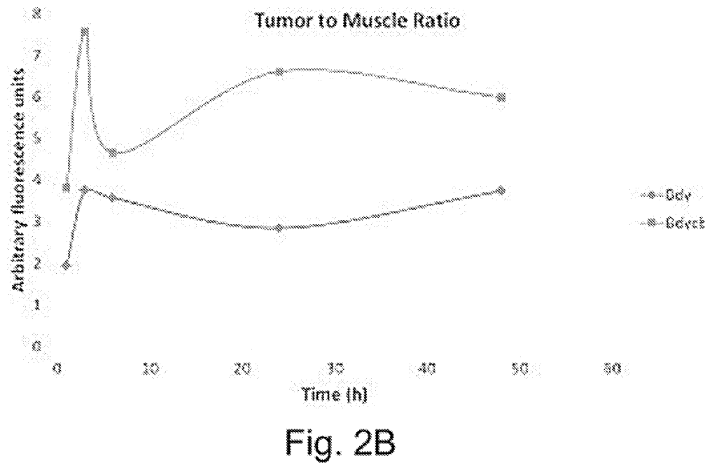

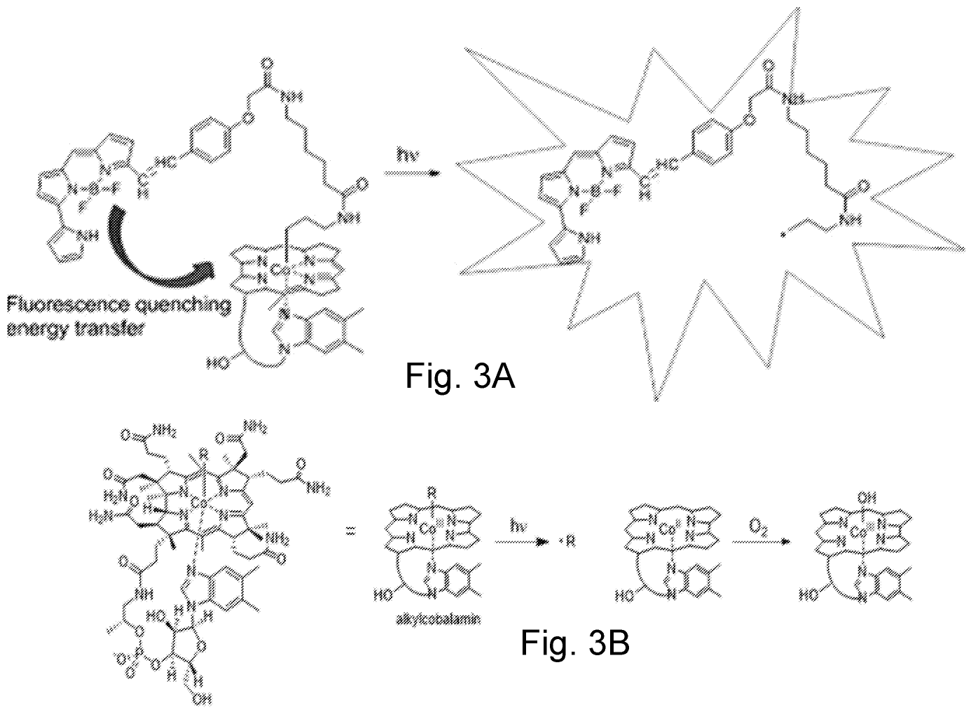

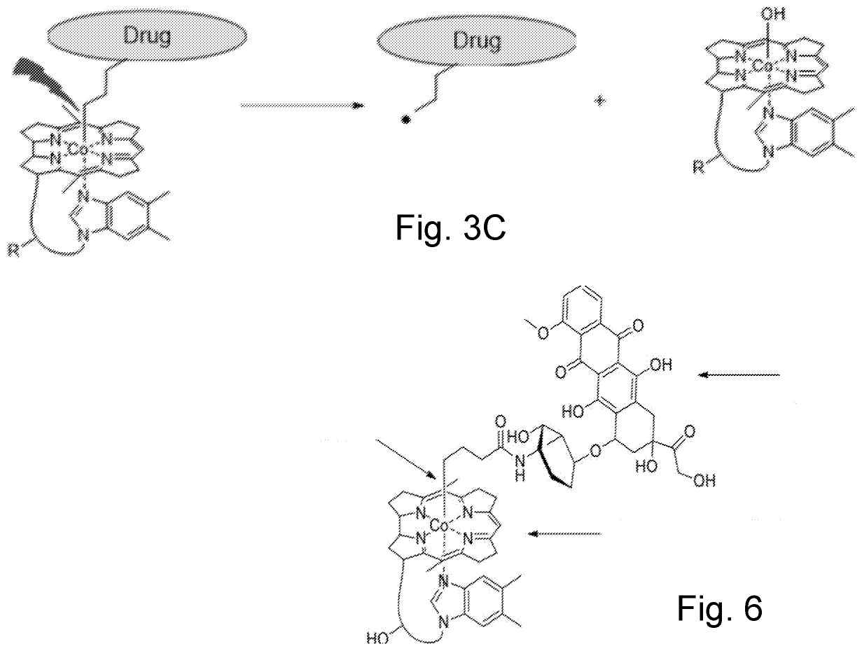

Bodipy650-labeled cobalamin derivative (Bdy-Cbl) (FIG. 2A) was synthesized by reducing hydroxocobalamin with Zn, and allowing it to react with 3-bromopropylamine to form aminopropyl cobalamin. Aminopropyl cobalamin was then allowed to react with Bodipy650-NHS ester to form the Bodipy-cobalamin.

In other embodiments, in place of bodipy650, one of tetramethylrhodamine, sulfocyanine 5 (otherwise known as Sulfo-Cy5), AlexaFluor700, Atto 725, IRDye700, or DyLight800 is used as the fluorophore.

Bdy-Cbl (100 micromolar) was injected via tail vein into athymic nude mice implanted with MCF-7 and MIA PaCa-2 tumors. Fluorescence imaging was performed in vivo and with the organs ex vivo at a series of time points spanning 24 h. The Bdy-Cbl accumulated selectively in both tumor types, with maximum localization occurring at 24 h for the MCF-7 tumors and 3 h for the MIA PaCa-2 tumors, respectively. Bodipy650-labeled cobalamin conjugate was shown to concentrate in both MCF-7 and MIA PaCa-2 tumors relative to muscle (FIG. 2B) in athymic nude mice, which both overexpress TCblR, demonstrating the effectiveness of the vitamin B12 scaffold as a targeting agent.

This Bdy-Cbl was also shown to localize in normal tissues of mice differently than Bodipy650 alone (Bdy). This acylcobalamin derivative is also shown to release the Bdy fluorophore from the cobalamin scaffold upon red light irradiation. Here we quantified the activating light dose at 51.9 mJ/cm² for drug-cobalamin release, which is well within normal clinical doses required for photodynamic therapy. We have shown that Bdy release can be accomplished with red light that penetrates tissue far better than does blue or ultraviolet light. This cobalamin derivative was also activated with clinical X-ray doses from a linear accelerator at 6 MeV energies, demonstrating potential for action as a radiation-induced photopharmaceutical. We have shown that the conjugate (in this experiment the Bdy fluorophore) can be released from the cobalamin platform at single-session X-ray doses as low as 0.2 Gray (Gy), with maximal release occurring at 2 Gy, a typical single dose for tumor radiotherapy. This cobalamin platform technology does not require light transfer from a nanoscintillator for drug activation. The drug is released directly with X-ray irradiation, allowing for in addition, the drug is released from the cobalamin scaffold at relatively low radiation doses such as 0.2 Gy, which could be achieved with higher X-ray CT doses.

While our cobalamin platform technology can be directly activated by X-ray irradiation, allowing consistent drug release at low radiation doses, addition of a fluorophore 110 (FIG. 1) to a cobalamin-drug conjugate improves sensitivity of release to radiation and, if correctly chosen, allows release of drug from the molecule to be triggered by light from a light-emitting diode (LED) as well as X-ray from a clinical linear accelerator. The fluorophore 110, when attached to the 5′ ribose hydroxyl group of the cobalamin, is stimulated by arriving X-ray or optical light 112, the fluorophore 110 then transfers energy to the light-cleavable bond 106. Selectivity of the cobalamin platform for tumors combined with its X-ray activatability promises high precision targeting utilizing traditional radiotherapy.

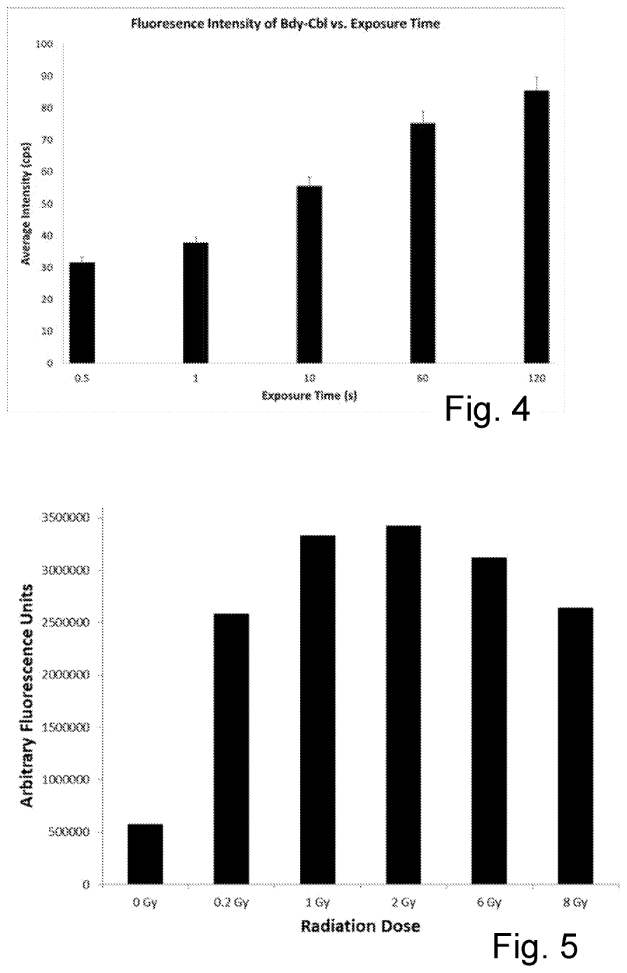

We also found that, because fluorescence of the Bdy is suppressed by energy transfer to the light-cleavable bond, fluorescence increases with light exposure (FIG. 4) as fluorophore is released by cleaving cobalt-carbon bonds from the Bdy-Cbl conjugate. This increase in fluorescence is a result of fluorescence quenching in Bdy-Cbl conjugate by the cobalamin corin ring that does not occur in free Bdy. Similarly, as illustrated in FIG. 5, fluorescence increases with X-ray exposure as fluorophore is released from the Bdy-Cbl conjugate until, at high radiation doses, fluorophore is destroyed by the radiation. This was confirmed with measurements of excretion because the unconjugated Bdy-carboxylate was excreted mostly through the liver, while the Bdy-Cbl was excreted primarily through the kidneys—the route of cobalamin excretion.

Not every drug can be attached to the cobalamin platform in a facile manner due to the nature of the functional groups on the drug. However, we have been able to attach a large library of drugs that are released in a light activatable fashion that are clinically relevant and have a variety of modes of action. Antineoplastic drugs we have attached include the DNA interference agent doxorubicin, the mitotic spindle inhibitor paclitaxel, the anti-folate methotrexate, the epidermal growth factor (EGFR) inhibitor erlotinib, the DNA alkylation agent chlorambucil, the tyrosine kinase inhibitor dasatinib, the DNA topoisomerase I inhibitor SN38, and the mitotic spindle disruptor colchicine, among others. We anticipate most small-molecule antineoplastic or antiviral drugs having an amine, alcohol, or carboxylic acid functional group can be conjugated to the cobalamin platform through that functional group and transported via transcobalamin into the cell.

We have demonstrated an effect on kinase activity in a light-dependent fashion. A cyclic adenosine monophosphate (cAMP) derivative was conjugated to the cobalamin scaffold (Cbl-cAMP). Upon irradiation with light, reorganization of the actin cytoskeleton was observed in cultured rat embryonic fibroblasts, a known result of the activation of protein kinase A pathway.

Most importantly, the cobalamin scaffold was conjugated to doxorubicin (Dox), a frequently utilized chemotherapeutic for a variety of cancers. Cobalamin-doxorubicin (Cbl-Dox) conjugate was synthesized from a carboxylic acid modified cobalamin platform and doxorubicin via a standard amide bond formation with coupling agent N,N,N′,N′-Tetramethyl-O-(1H-benzotriazol-1-yl)uronium hexafluorophosphate, O-(Benzotriazol-1-yl)-N,N,N′,N′-tetramethyluronium hexafluorophosphate (HBTU, FIG. 6). We examined the cytotoxicity of Cbl-Dox in HeLa cells in the dark and in the presence of increasing light illumination times. Cbl-Dox exposure in the absence of light is innocuous. In contrast, treatment with Cbl-Dox and light demonstrates a light-dose dependent increase in cell mortality, which with high light exposure matches that of doxorubicin alone.

### PDAC Treatment

We found that Cbl-Bdy selectively targets MIA PaCa-2 pancreas tumors versus normal pancreas at an average of 2.4:1 ratio (FIG. 6), with maximum uptake occurring at 48 hours post injection. This is a remarkable discovery for drug delivery to pancreas tumors, as this is one of the most difficult challenges to treating PDAC. PDAC tumors lack vascularization and contain large amounts of collagen, making them stiff and difficult to treat.

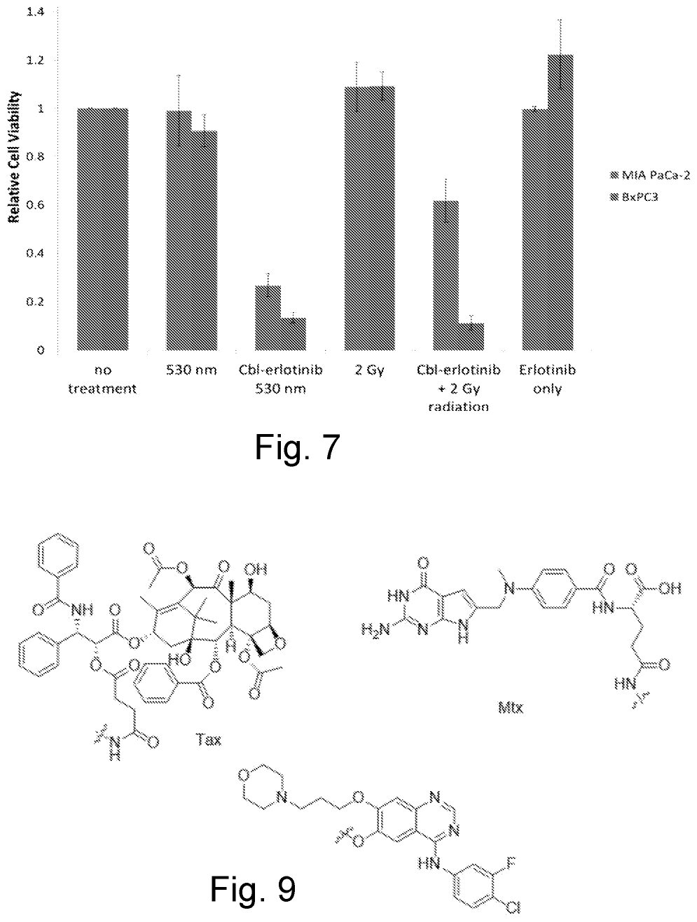

We investigated a cobalamin-erlotinib (Cbl-Erl) conjugate in two pancreatic cancer cultured cell lines previously demonstrated to overexpress TCblR, these cell lines are MIA PaCa-2 and BxPC3. These cells were treated with Cbl-Erl (1 micromolar) and given a single radiation dose of 2 Gy. Irradiation was also performed at 530 nm, within the natural absorbance of the cobalamin, as a positive control. There is radiation-dependent cell killing in both cell lines with the combination of Cbl-Erl and a 2 Gy dose from the linear accelerator or light at 530 nm (FIG. 7). Radiation alone did not cause significant apoptosis. Illumination with 530 nm light alone exhibited no significant cell death. The cobalamin-erlotinib (Cbl-Erl) conjugate activated by either 530 nm. visible light as a positive control or X-ray radiation exhibits significantly greater toxicity to MIA PaCa-2 (left bars) (FIG. 7) and BxPC3 (right bars) mouse pancreatic cancer cells than light, X-ray, or unconjugated erlotinib alone. This indicates the light-or-X-ray-activated, conjugated, drug-cobalamin treatment is likely more effective against such tumors than the drug, light, or X-ray treatment alone, and we are conducting in-vivo studies. We are conducting similar studies with a Cbl-SN-38 conjugate.

We have synthesized the SN38 and erlotinib conjugates without the Cy5 handle, using the synthesis described, and we are evaluating them in vitro as described herein

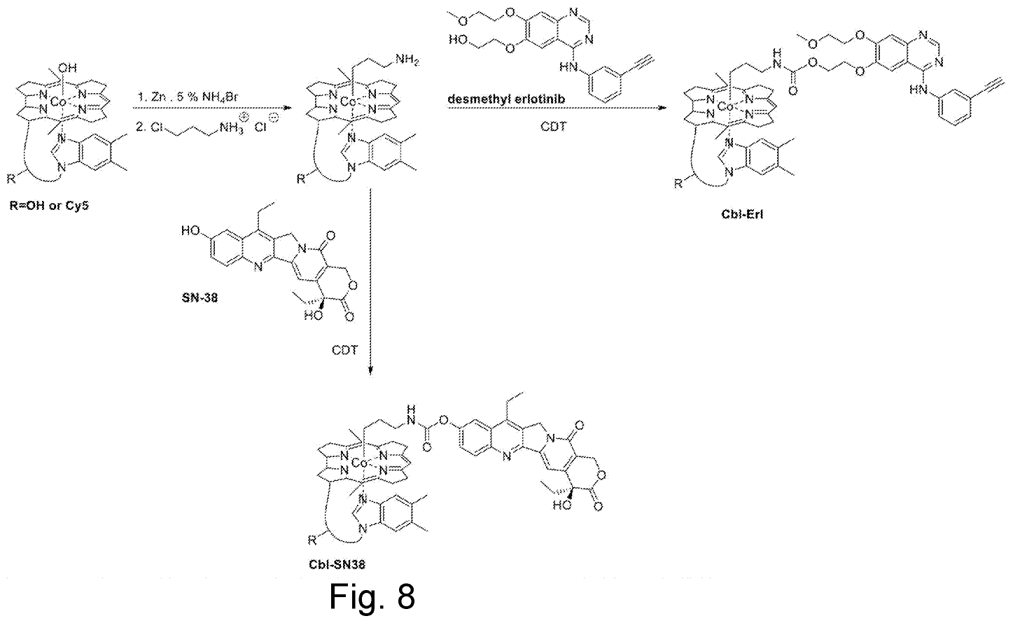

In addition to the doxorubicin-cobalamin conjugate above (Cbl-Dox), SN-38, the more potent metabolite of irinotecan, and erlotinib will be linked to the cobalamin scaffold by conjugation through a hydroxyl group to an aminopropyl cobalamin intermediate via carbamate formation through standard coupling with 1,1′-Carbonyl-di-(1,2,4-triazole) (CDT). The conjugate will also be synthesized with a Cy5 fluorescent handle, which is accomplished via coupling of the 5′ hydroxyl group of the ribose first with ethylene diamine and then with the N-hydroxysuccinimide of the fluorophore (Cy5). See FIG. 8. This will form cobalamin-Cy5-erlotinib (Cbl-Cy5-Erl) and cobalamin-Cy5-SN38 (Cbl-Cy5-SN38). The Cy5 handle allows for the red and NIR-activation of the chemotherapeutics as well as for the ability to visualize distribution of the drugs in tumors. We have readily and reproducibly attached this fluorophore multiple times. In an alternative embodiment, a synthetic strategy based on linkage to the cobalamin platform via a Williamson ether synthesis with a cobalamin containing a haloalkane handle.

We have determined in preliminary studies that the drug can be released from the cobalamin platform at clinically relevant PDT doses, so we will do a more extensive evaluation for each specific drug-cobalamin conjugate with both visible (530 nm), and X-ray irradiation in vitro as a control prior to utilizing these compounds in conjunction with radiation therapy in vivo.

In an alternative embodiment, we add transcobalamin II to enhance uptake of cobalamin derivatives into tissue. In another alternative embodiment, the cobalamin scaffold is linked to nanoparticles, which have been shown to enhance permeability and retention. Drug delivery is thought to be impeded by the dense stroma of pancreatic adenocarcinoma, and so delivery of cobalamin conjugates could be limited by this issue. Small molecular therapeutics such as Losartan can reduce stromal formation and allow increases in microvessel perfusion, and preliminary data in our laboratory has confirmed this in two tumor models. Thus, we believe that a combination of cobalamin with neoadjuvant Losartan is a viable pathway to increase delivery and thereby maximize effect of the light/radiation activated cobalamin. In an alternative embodiment we treat with Losartan for 10 days prior to cobalamin-drug conjugate treatment to enhance the delivery of this platform to the PDAC tumor stroma.

The added benefit of the cobalamin platform will be assessed in comparison to radiation therapy, using the range of control groups required to assess synergy of interaction. Mice with subcutaneous PDAC tumors injected in the flank using cell lines described above (MIA PaCa-2 and BxPC3 in the range of 125-175 mm3) will be used for these studies randomly chosen from each cage, both male and female, with care to ensure that there are matched sizes in each group. Mice will be anesthetized by inhaled isoflurane to maintain blood flow and tumor oxygenation, and supported on an electric heating pad and temperature monitored with a rectal probe. The tumors will be irradiated with a 6 MeV electron beam using a 5 mm cone, and with a 1 cm bolus over the tumor area, to ensure Dmax is reached at the point of the tumor. Treatment planning will be completed for the mice range of tumor sizes and a cone-beam CT will be completed for each mouse to verify position on the bed. A dose of 5 Gy/day for 5 days will be given to the tumor in each case. If this dose exhibits toxicity in the mice, we may opt for a lower hypofractionated dose, however we have used scheme in the past for H&N tumors in mice and it has worked well to be sub-curative but effective in tumor shrinkage. This matches some trials used for pancreatic cancer, and matches some SBRT applications reasonably well. We plan to assess all compounds listed above for their ability to cause a synergistic effect with radiation.

Tumor size in a rodent model of PDAC will be assessed and compared under these variety of conditions:

1. Combination of Cbl-Dox, Cbl-SN38, and Cbl-Erl to assess the synergistic effect of these light-activated chemotherapeutics together (n=16 animals)

2. Combination of Cbl-Dox and radiation therapy (n=16 animals)

3. Combination of Cbl-SN38 and radiation therapy (n=16 animals)

4. Combination of Cbl-Erl and radiation therapy (n=16 animals)

5. Combination Dox and radiation therapy (n=16 animals)

6. Combination of SN-38 and radiation therapy (n=16 animals)

7. Combination of erlotinib and radiation therapy (n=16 animals)

8. Control group radiation therapy alone (n=16 animals)

9. Control group of Cbl-Dox alone (n=16 animals each)

10. Control group of Cbl-Erl alone (n=16 animals each)

11. Control group of Cbl-SN38 alone (n=16 animals each)

12. Control group no treatment (n=16 animals)

Data analysis & Statistics: Animals will be incubated with the compound for a period of days, as determined based upon our studies, and after approximately 2 weeks or 3 doubling periods of the control group, all animals will be sacrificed and PDAC tumors resected and sliced and stained for analysis. The tumor regrowth data will be analyzed in three ways, first by simple tumor regrowth assay, normalizing data relative to the starting size, and averaging results to assess doubling times. This will be the primary assay, and secondarily we will assess differences in tumor size at static times after treatment. Finally, if the tumors grow faster than expected, we will plot all data in a Kaplan-Meyer curve, assessing survival as a function of days, looking for differences between survival in the different treatment groups.

Ex Vivo Assays: We will assay a few tumors from each group during treatment to determine the effect upon the extent of the margin and microinvasion. This will be quantified by computer analysis of Ki67 images and H&E images, to determine which treatments were optimal.

We will investigate the root cause of the synergy of X-ray treatment with cobalamin-linked antineoplastic drugs such as Cbl-Dox, Cbl-SN38, Cbl-Erl, Cbl-Cy5-Dox, Cbl-Cy5-SN38, and Cbl-Cy5-Erl, by varying the Cherenkov light to radiation dose ratio of the therapy. This can be achieved by changing the beam energy while keeping the delivered dose fixed. We will repeat the best combination in the treatment groups above, using 18 MeV beam energy, which will have twice the Cherenkov light generation, but at the same delivered radiation dose to the tumor. In this way, we will be able to assess if Cherenkov light is a dominant factor in enhancing the tumor killing effect or the radiation dose acting directly with the carbon-cobalt bond of the agent.

### The Method Reviewed

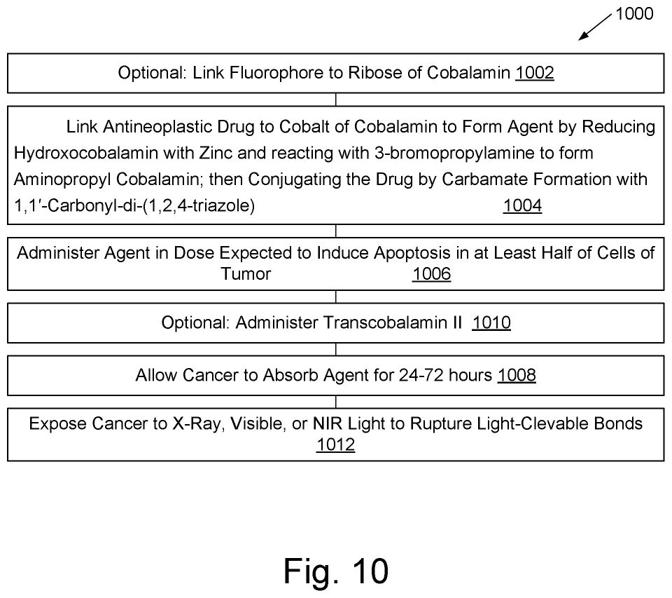

As illustrated in FIG. 10, a method 1000 of treating cancer, such as PDAC, begins with, if high sensitivity to radiation is desired, linking 1002 a fluorophore, such as Cy5, to the 5′ hydroxyl of ribose of hydroxycobalamin. The antineoplastic drug is linked 1004 to cobalt of cobalamin to form the agent herein described by reducing hydroxocobalamin with zinc and reacting with 3-bromopropylamine to form aminopropyl cobalamin; then conjugating the drug by carbamate formation with 1,1′-carbonyl-di-(1,2,4-triazole). The agent is then administered 1006 to the subject in a dose sufficient to induce apoptosis in at least half of cells of the cancer once the light-cleavable bonds are cleaved, and allowed to absorb 1008 into the tumor for between 24 and 72 hours, and ideally about 48 hours. In an alternative embodiment, the agent is coadministered 1010 with transcobalamin II. The cancer, and agent therein, is then exposed 1012 to X-ray or visible light to cleave the radiation-and-light-cleavable bonds between cobalt of the cobalamin and the antineoplastic drug, releasing the antineoplastic drug to kill the cancer cells. In an embodiment, the X-ray or visible light is X-ray sufficiently intense to kill many cells of the cancer.

### Combinations:

The methods and compounds herein described can be combined in many ways. Among ways anticipated by the inventors are:

A therapeutic agent designated A for radiation-activated chemotherapy including an antineoplastic drug and a cobalamin; the antineoplastic drug bonded with a light-cleavable bond to a cobalt of the cobalamin.

A therapeutic agent designated AA including the therapeutic agent designated A where the antineoplastic drug is doxorubicin, paclitaxel, methotrexate, erlotinib, chlorambucil, dasatinib, SN38, colchicine, or gefitinib.

A therapeutic agent designated AB including the therapeutic agent designated A or AA further comprising a fluorophore bonded to a ribose of the cobalamin.

A therapeutic agent designated AC including the therapeutic agent designated AB where the fluorophore is bonded by substitution of a hydrogen at a 5′ hydroxyl group of the ribose of the cobalamin.

A therapeutic agent designated AD including the therapeutic agent designated A, or AA further including a fluorophore bonded by substitution of a hydrogen at a 5′ hydroxyl group of a ribose of the cobalamin.

A therapeutic agent designated AE including the therapeutic agent designated AB, AC, or AD where the fluorophore is Cy5.

A therapeutic agent designated AF including the therapeutic agent designated A, AA, AB, AC, AD, or AD and further comprising transcobalamin II.

A therapeutic agent designated AG comprising the therapeutic agent designated A wherein the antineoplastic drug is a small-molecule antineoplastic drug having an amine, alcohol, or carboxylic acid functional group conjugated to a cobalt of the cobalamin.

A therapeutic agent designated AH comprising the therapeutic agent designated A, AA, or AG further comprising a fluorophore selected from the group consisting of tetramethylrhodamine, AlexaFluor700, Atto 725, IRDye700, and DyLight800 conjugated to the cobalamin.

A therapeutic agent designated AJ including the therapeutic agent designated AH wherein the fluorophore is conjugated to the cobalamin at a 5′ hydroxyl group of a ribose residue of the cobalamin.

A method of preparing a therapeutic agent designated B including bonding, with a light-cleavable bond, an antineoplastic agent to a cobalt atom of a cobalamin.

A method of preparing a therapeutic agent designated BA including the method designated B further including bonding a fluorophore to a ribose of the cobalamin.

A method of preparing a therapeutic agent designated BB including the method designated B or BA wherein the antineoplastic drug is selected from the group consisting of doxorubicin, paclitaxel, methotrexate, erlotinib, chlorambucil, dasatinib, SN38, colchicine, and gefitinib.

A method of preparing a therapeutic agent designated BC including the method designated B, BA, BB, or BF further including bonding a fluorophore to a ribose of the cobalamin.

A method of preparing a therapeutic agent designated BD including the method designated BC where the fluorophore is Cy5.

A method of preparing a therapeutic agent designated BE including the method designated B, BA, BB, BC, or BD further including adding transcobalmin II.

A method of preparing a therapeutic agent designated BF including the method designated B or BA wherein the antineoplastic drug is a small-molecule antineoplastic drug having an amine, alcohol, or carboxylic acid functional group conjugated to a cobalt of the cobalamin.

A method of preparing a therapeutic agent designated BG including the method designated BC where the fluorophore is selected from the group consisting of tetramethylrhodamine, AlexaFluor700, Atto 725, IRDye700, and DyLight800 conjugated to the cobalamin.

A therapeutic agent designated AJ including the therapeutic agent designated AH wherein the fluorophore is conjugated to the cobalamin at a 5′ hydroxyl group of a ribose residue of the cobalamin.

A method of forming a cobalamin-drug conjugate designated C including: reducing hydroxocobalamin with zinc, and allowing the reduced hydroxocobalamin to react with 3-bromopropylamine to form aminopropyl cobalamin; and linking an antineoplastic drug to the aminopropyl cobalamin by conjugation through a hydroxyl group to the aminopropyl cobalamin by carbamate formation with 1,1′-Carbonyl-di-(1,2,4-triazole).

A method of forming a cobalamin-drug conjugate designated CA including the method designated C wherein the antineoplastic drug is selected from the group consisting of doxorubicin, paclitaxel, methotrexate, erlotinib, chlorambucil, dasatinib, SN38, colchicine, and gefitinib.

A method of forming a cobalamin-drug conjugate designated CB including the method designated C or CA further including adding a Cy5 fluorescent handle to a ribose of the aminopropyl cobalamin by coupling a 5′ hydroxyl group of the ribose first with ethylene diamine and then with the N-hydroxysuccinimide of Cy5.

A method of treatment of a cancer in a mammal designated D including: administering the therapeutic agent designated A, AA, AB, AC, AD, AE, or AF to the mammal in a dose expected to induce apoptosis in a majority of cells of the cancer when the light-cleavable bond is cleaved; allowing the cancer to absorb the therapeutic agent; exposing the cancer to radiation selected from the group consisting of X-ray radiation, visible light, and near-infrared light sufficient to cleave the light-cleavable bond between the antineoplastic drug and the cobalt of the cobalamin.

A method of treatment of a cancer designated DA including the method designated D where the cancer is a pancreatic ductal adenocarcinoma.

A method of treatment of a cancer designated DB including the method designated D or DA where the mammal is a human.

A method of treatment of a cancer designated DC including the method designated D or DA further comprising administering transcobalamin II to the mammal concurrently with the therapeutic agent.

### CONCLUSION

This cobalamin technology platform provides precision targeting of chemotherapy in conjunction with traditional radiotherapy, which is something that has been difficult to achieve. This invention can be applied to transform traditional chemotherapeutics into tumor-targeted, visible or X-ray radiation-activated, chemotherapeutic agents.

Changes may be made in the above system, methods or device without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A therapeutic agent for radiation-activated chemotherapy comprising:
an antineoplastic drug; and
a cobalamin;
the antineoplastic drug bonded with an light or X-ray cleavable bond to a cobalt of the cobalamin.

2. The therapeutic agent of claim 1 wherein the antineoplastic drug is selected from the group consisting of doxorubicin, paclitaxel, erlotinib, chlorambucil, dasatinib, SN38, colchicine, and gefitinib.

3. The therapeutic agent of claim 1 further comprising a fluorophore bonded to a ribose of the cobalamin.

4. The therapeutic agent of claim 3 wherein the fluorophore is bonded by substitution of a hydrogen at a 5′ hydroxyl group of the ribose of the cobalamin.

5. The therapeutic agent of claim 2 further comprising a fluorophore bonded by substitution of a hydrogen at a 5′ hydroxyl group of a ribose of the cobalamin.

6. The therapeutic agent of claim 3 where the fluorophore is Sulfo-Cy5.

7. The therapeutic agent of claim 6 further comprising transcobalamin II.

8. The therapeutic agent of claim 2, where the cobalamin is vitamin B-12.

9. The therapeutic agent of claim 2, where the cobalamin is an alkylcobalamin.

10. A method of preparing a therapeutic agent comprising:
bonding, with a light-cleavable bond, an antineoplastic agent to a cobalt atom of a cobalamin.

11. The method of claim 10 further comprising bonding a fluorophore to a ribose of the cobalamin.

12. The method of claim 10 wherein the antineoplastic drug is selected from the group consisting of doxorubicin, paclitaxel, methotrexate, erlotinib, chlorambucil, dasatinib, SN38, colchicine, and gefitinib.

13. The method of claim 12 further comprising bonding a fluorophore to a ribose of the cobalamin.

14. The method of claim 13 where the fluorophore is Cy5.

15. The method of claim 14 further comprising adding transcobalmin II.

16. A method of forming a cobalamin-drug conjugate comprising:
reducing hydroxocobalamin with zinc, and allowing the reduced hydroxocobalamin to react with 3-bromopropylamine to form aminopropyl cobalamin;
linking an antineoplastic drug to the aminopropyl cobalamin by conjugation through a hydroxyl group to the aminopropyl cobalamin by carbamate formation with 1,1′-Carbonyl-di-(1,2,4-triazole).

17. The method of claim 16 wherein the antineoplastic drug is selected from the group consisting of doxorubicin, paclitaxel, methotrexate, erlotinib, chlorambucil, dasatinib, SN38, colchicine, and gefitinib.

18. The method of claim 16 further comprising adding a Cy5 fluorescent handle to a ribose of the aminopropyl cobalamin by coupling a 5′ hydroxyl group of the ribose first with ethylene diamine and then with the N-hydroxysuccinimide of Cy5.

19. A method of treatment of a cancer in a mammal comprising:
administering the therapeutic agent of claim 2, to the mammal in a dose expected to induce apoptosis in a majority of cells of the cancer when the light-cleavable bond is cleaved;
allowing the cancer to absorb the therapeutic agent; and
exposing the cancer to radiation selected from the group consisting of X-ray radiation, visible light, and near-infrared light sufficient to cleave the light-cleavable bond between the antineoplastic drug and the cobalt of the cobalamin.

20. The method of treatment of a cancer of claim 19 wherein the cancer is a pancreatic ductal adenocarcinoma.

21. The method of treatment of cancer of claim 20 where the mammal is a human.

22. The method of treatment of cancer of claim 21 further comprising administering transcobalamin II to the mammal.

23. The method of treatment of cancer of claim 22 wherein the cancer is allowed to absorb the therapeutic agent for at least 24 hours.

24. The therapeutic agent of claim 1 wherein the antineoplastic drug is a small-molecule antineoplastic drug having an amine, alcohol, or carboxylic acid functional group conjugated to a cobalt of the cobalamin.

25. The therapeutic agent of claim 1, further comprising a fluorophore selected from the group consisting of tetramethylrhodamine, AlexaFluor700, Atto 725, IRDye700, and DyLight800 conjugated to the cobalamin.

26. The therapeutic agent of claim 25 wherein the fluorophore is conjugated to the cobalamin at a 5′ hydroxyl group of a ribose residue of the cobalamin.
